(19) United States
(12) Patent Application Publication
(10) Pub. No.: US 2024/0382732 A1
(43) Pub. Date: Nov. 21, 2024

(54) Multi-Lumen Microneedle For Simultaneous Intracochlear Injection And Aspiration

(71) Applicant: The Trustees of Columbia University in the City of New York, New York, NY (US)

(72) Inventors: Aykut AKSIT, New York, NY (US); Stephen LEONG, New York, NY (US); Anil K. LALWANI, New York, NY (US); Jeffrey W. KYSAR, New York, NY (US)

(21) Appl. No.: 18/784,013

(22) Filed: Jul. 25, 2024

Related U.S. Application Data
(63) Continuation of application No. PCT/US2023/061767, filed on Feb. 1, 2023.
(60) Provisional application No. 63/305,551, filed on Feb. 1, 2022.

(51) Int. Cl. A61M 37/00; A61M 5/178

(52) U.S. Cl. CPC A61M 37/0015; A61M 5/178

(57) ABSTRACT

A microneedle system is provided including a needle assembly having a microneedle with a longitudinal body with a diameter of about 50 microns to about 200 microns, the microneedle defining a sharpened tip and a proximal end thereof, the microneedle having first and second lumens extending therethrough, each of the first and second lumens having a distal openings located proximal to the sharpened tip and a proximal opening at a proximal end of the microneedle; a base having a distal end and a proximal end, the distal end of the base coupled to the proximal end of the microneedle and defining first and second passages in fluid communication with the first and second lumens; and a pair of tubes coupled to the base, each tube in fluid communication with a respective first and second passage of the base; a syringe pump; and circulation tubing connecting the syringe pump with the pair of tubes in the needle assembly.

### CROSS-REFERENCE TO RELATED APPLICATION(S)

This application is a continuation of International Application No. PCT/US2023/061767, filed Feb. 1, 2023; which application claims priority to U.S. Provisional Application 63/305,551, filed Feb. 1, 2022. All foregoing applications are incorporated herein by reference in their entireties for any and all purposes.

### GOVERNMENT SUPPORT CLAUSE

This invention was made with government support under grant DC014547 awarded by the National Institutes of Health. The government has certain rights in the invention.

### FIELD

The disclosed subject matter relates to a microneedle for delivery and aspiration of therapeutic agents. More particularly, the subject matter relates to a microneedle configured to deliver a dosing of therapeutic agent and aspiration of fluid from an anatomic compartment.

### BACKGROUND

Delivery of a precise dose of medication across the anatomic barriers, such as for example to the Central Nervous System (CNS), is a serious challenge for clinicians of multiple specialties. For example, the current methods of therapeutic delivery to the cochlea are inherently imprecise and can result in functional damage to the auditory and vestibular systems. The microneedle embodied herein allows for controlled delivery of therapeutic agents across barrier tissues via temporary microscopic perforations induced by at least one microscopic needle, and the delivery of a cleavable portion of the microneedle body containing a precise amount or dose of therapeutic agent.

Delivery of a precise dose of medication across other anatomic barriers to the Central Nervous System (CNS) is also a serious challenge for clinicians of multiple specialties. Thus, there is a need for a microneedle for local delivery of therapeutic agents across anatomic membranes that is reliable and predictable without promoting anatomic or functional damage.

### SUMMARY

In one aspect, the disclosed subject matter provides a microneedle system including a needle assembly having a microneedle with a longitudinal body with a diameter of about 50 microns to about 200 microns, the microneedle defining a sharpened tip and a proximal end thereof, the microneedle having first and second lumens extending therethrough, each of the first and second lumens having a distal openings located proximal to the sharpened tip and a proximal opening at a proximal end of the microneedle; a base having a distal end and a proximal end, the distal end of the base coupled to the proximal end of the microneedle and defining first and second passages in fluid communication with the first and second lumens; and a pair of tubes coupled to the base, each tube in fluid communication with a respective first and second passage of the base; a syringe pump; and circulation tubing connecting the syringe pump with the pair of tubes in the needle assembly.

In some embodiments, the microneedle has a maximum outer diameter less than about 100 microns along a portion of the longitudinal body. In some embodiments, the microneedle has a maximum outer diameter less than about 50 microns along a portion of the longitudinal body. In some embodiments, the microneedle is fabricated from a biocompatible polymers, stainless steel, or titanium. In some embodiments, the base is integral with the microneedle.

In some embodiments, the pair of tube includes a blunt metallic syringe needle and a flexible polymer tubing. In some embodiments, the flexible polymer tubing is fabricated from polyimide material. In some embodiments, the blunt metallic syringe needle is a 30-gauge syringe needle. In some embodiments, the pair of tube comprising two blunt metallic syringe needles.

In some embodiments, the first and second lumens have a diameter of about 15 microns to about 80 microns. In some embodiments, the first and second lumens have a diameter of about 30 microns.

In some embodiments, fluid is aspirated into one of the first and second lumens at a flow rate of a maximum of about 0.12 microliters per second. In some embodiments, fluid is injected from one of the first and second lumens at a flow rate of a maximum of about 0.20 microliters per second.

In some embodiments, the microneedle has a length of about 250 microns to about 750 microns. In some embodiments, the microneedle has a length of about 475 microns.

In some embodiments, the first and second distal lumens opening are aligned along the length of the microneedle. In some embodiments, the first and second distal lumen openings are offset along the length of the needle.

In some embodiments, the base defines first and second concentric passages for providing fluid communication between the first and second lumens and the pair of tubes.

In another aspect, the disclosed subject matter provides s microneedle system including a needle assembly having: a first microneedle with a longitudinal body with a diameter of about 50 microns to about 200 microns, the microneedle defining a sharpened tip and a proximal end thereof, the first microneedle having first lumen extending therethrough, the first lumen having a distal opening located proximal to the sharpened tip and a proximal opening at a proximal end of the first microneedle; a second microneedle having a longitudinal body with a diameter of about 50 microns to about 200 microns, the second microneedle defining a sharpened tip and a proximal end thereof, the microneedle having second lumen extending therethrough, the second lumen having a distal opening located proximal to the sharpened tip and a proximal opening at a proximal end of the second microneedle; a base having a distal end and a proximal end, the distal end of the base coupled to the proximal end of the first microneedle and the proximal end of the second microneedle, the base defining first and second passages in fluid communication with the first and second lumens; and a pair of tubes coupled to the base, each tube in fluid communication with a respective first and second passage of the base; a syringe pump; and circulation tubing connecting the syringe pump with the pair of tubes in the needle assembly.

In some embodiments, the first and second microneedles have a maximum outer diameter less than about 100 microns along a portion of the longitudinal body. In some embodiments, the first and second microneedles have a maximum outer diameter less than about 50 microns along a portion of the longitudinal body.

In some embodiments, the first and second microneedles are fabricated from a biocompatible polymers, stainless steel, or titanium.

In some embodiments, the base is integral with first and second microneedles.

In some embodiments, the pair of tubes includes a blunt metallic syringe needle and a flexible polymer tubing. In some embodiments, the flexible polymer tubing is fabricated from polyimide material. In some embodiments, the blunt metallic syringe needle is a 30 gauge syringe needle. In some embodiments, the pair of tubes comprising two blunt metallic syringe needles.

In some embodiments, the first and second lumens each have a diameter of about 15 microns to about 80 microns. In some embodiments, the first and second lumens each have a diameter of about 30 microns.

In some embodiments, fluid is aspirated into one of the first and second lumens at a flow rate of a maximum of about 0.15 microliters per second. In some embodiments, fluid is injected from one of the first and second lumens at a flow rate of a maximum of about 0.2 microliters per second.

In some embodiments, the first and second microneedles have a length of about 250 microns to about 750 microns. In some embodiments, the first and second microneedles have a length of about 475 microns.

In some embodiments, the microneedle assembly further includes a third microneedle having a longitudinal body with a diameter of about 50 microns to about 200 microns, the third microneedle defining a sharpened tip and a proximal end thereof, the third microneedle having third lumen extending therethrough, the third lumen having a distal opening located proximal to the sharpened tip and a proximal opening at a proximal end of the third microneedle; wherein the base coupled to the proximal end of the third microneedle and defining a third passages in fluid communication with the third lumens.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is simplified representation of the anatomy of the human ear.

FIG. 2 is a simplified schematic view of microneedle system in accordance with an exemplary embodiment of the disclosed subject matter.

FIG. 3(a) is a cross-sectional view of microneedle system in accordance with a first exemplary embodiment of the disclosed subject matter.

FIG. 3(b) is a side view of microneedle system in accordance with a first exemplary embodiment of the disclosed subject matter.

FIG. 4 is a photograph of a portion of the microneedle system of FIGS. 3(a)-(b).

FIG. 5 is a cross-sectional view of a microneedle system in accordance with a second exemplary embodiment of the disclosed subject matter.

FIG. 6 is a side view of the microneedle system in accordance with the second exemplary embodiment of the disclosed subject matter.

FIG. 7 is a top view of a microneedle system in accordance with a third exemplary embodiment of the disclosed subject matter.

FIG. 8 is a cross-sectional view of the microneedle system in accordance with the third exemplary embodiment of the disclosed subject matter.

FIG. 9 is a top view with internal components shown in dashed line of a microneedle system in accordance with a fourth exemplary embodiment of the disclosed subject matter.

FIG. 10 is a cross-sectional view of the microneedle system in accordance with the fourth exemplary embodiment of the disclosed subject matter.

FIG. 11 is a side view with internal components shown in dashed line of the microneedle system in accordance with the fourth exemplary embodiment of the disclosed subject matter.

FIG. 12 is a side view of a microneedle system in accordance with a fifth exemplary embodiment of the disclosed subject matter.

FIG. 13 is a cross-sectional view of the microneedle system in accordance with the fifth exemplary embodiment of the disclosed subject matter.

FIG. 14 is a cross-sectional view of a microneedle system in accordance with a sixth exemplary embodiment of the disclosed subject matter.

FIG. 15 is a cross-sectional view of a microneedle system in accordance with a seventh exemplary embodiment of the disclosed subject matter.

FIG. 16 is perspective view of a microneedle system in accordance with an eighth exemplary embodiment of the disclosed subject matter.

FIG. 17 is a cross-sectional view of the microneedle system in accordance with the eighth exemplary embodiment of the disclosed subject matter.

### DETAILED DESCRIPTION

It is to be understood that both the foregoing general description and the following detailed description are exemplary and explanatory only, and are not restrictive of the invention, as claimed. In this description, the use of the singular includes the plural, the word “a” or “an” means “at least one,” and the use of “or” means “and/or,” unless specifically stated otherwise. Furthermore, the use of the term “including,” as well as other forms, such as “includes” and “included” is not limiting. Also, terms such as “element” or “component” encompass both elements or components comprising one unit and elements or components that comprise more than one unit unless specifically stated otherwise.

Use of the term “about,” when used with a numerical value, is intended to include +/−10%. For example, if a dimension is identified as about 200, this would include 180 to 220 (plus or minus 10%).

The terms “patient,” “individual,” and “subject” are used interchangeably herein, and refer to a mammalian subject to be treated, with human patients being preferred. In some cases, the methods of the invention find use in experimental animals, in veterinary application, and in the development of animal models for disease.

A microneedle apparatus is disclosed herein that facilitates simultaneous aspiration and injection of fluids into an anatomic compartment.

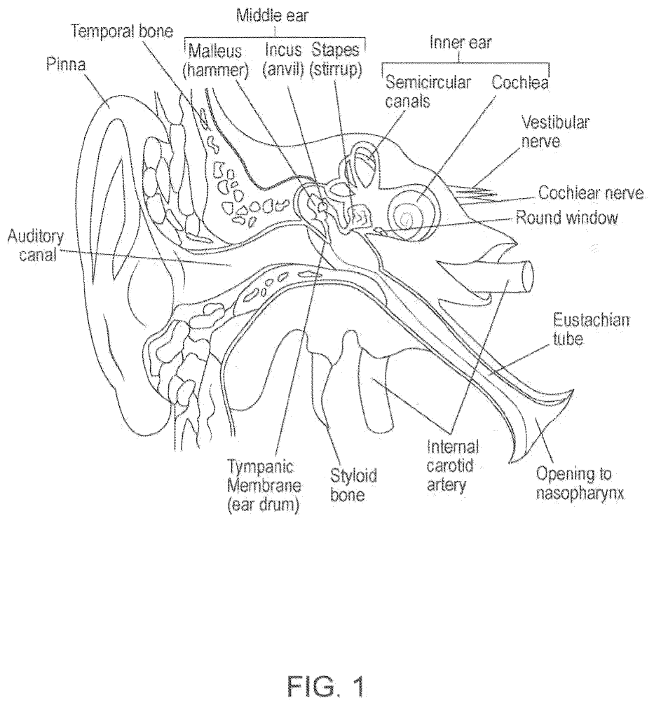

As shown in FIG. 1, the anatomy of the ear includes a middle ear comprising the hammer, anvil, and stirrup bones, and an inner ear comprising the semicircular canals and cochlea. The middle ear and inner ear have barriers to entry and are separated from auditory canal by the tympanic membrane or ear drum. Moreover, the inner ear is further protected from entry by its almost impenetrable structure. The RWM (secondary tympanic membrane) disposed at the inner ear provides an avenue to permit local delivery of therapeutic agents directly to the inner ear.

The RWM is a three-layered structure designed to protect the inner ear from middle ear pathology and facilitate active transport. There is an outer epithelial layer that faces the middle ear, a central connective tissue layer, and an inner epithelial layer interfacing with the scala tympani. The most prominent feature of the outer epithelial layer is the extensive interdigitations and tight junctions of its cells; in addition, there is also a continuous basement membrane layer. This architecture with tight junctions and a continuous basement membrane functions as a defensive shield designed to protect the inner ear from middle ear infections. The connective tissue core contains fibroblasts, collagen, and elastic fibers, and houses blood and lymph vessels. The connective tissue is divided roughly into thirds differing in fiber type and density thus essentially establishing a gradient. This layer is responsible for providing compliance to the RWM. Finally, there is a discontinuous inner epithelial layer that bathes in the perilymph of the scala tympani. As previously noted, conventional transtympanic delivery is limited as it relies on the ability of particles to diffuse or be actively transported across this three layered membrane.

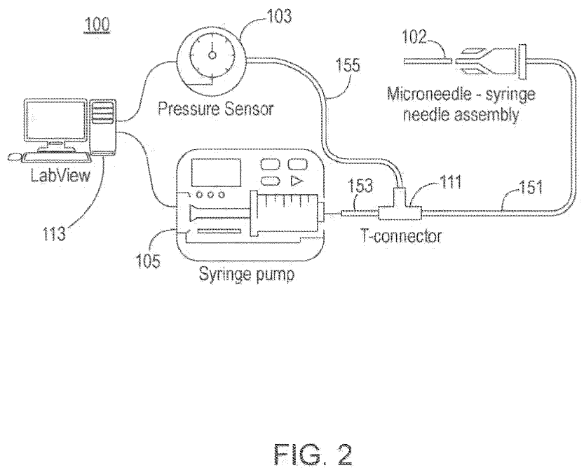

Microneedle System. The microneedle system 100 illustrated in FIG. 2 and described herein allows for the simultaneous intracochlear injection and aspiration. In an exemplary embodiment, the system 100 includes the microneedle assembly 102, a pressure sensor 103, a syringe pump 105, and a T-connector 111. Microneedle assembly 102 is fluidly connected to T-connector 111 via tubing 151. Pressure sensor 103 is connected to T-connector 111 via tubing 155. Syringe pump 105 is connected to T-connector 111 via tubing 153. PID control was provided by a computer 113 operating control software.

The microneedle assembly 102 includes a microneedle 104 described herein. Microneedle 104 was mounted on the tips of two blunt syringe needles 124a/b that, in turn, are connected to a syringe pump 105. In some embodiments, an exemplary syringe pump is Era pump systems, NE-1000 programmable pump. The syringe pump 105 was used to control the pressure leading to the microneedle assembly 102. A pressure transducer is used (not shown), for example Omega PX409-015GUSBH. Control software, such as LabView, was used for PID control. In some embodiments, the pressure sensor 103 and the microneedle assembly 102 were kept at the same level to be able to measure the correct pressure being applied between the microneedle and ambient.

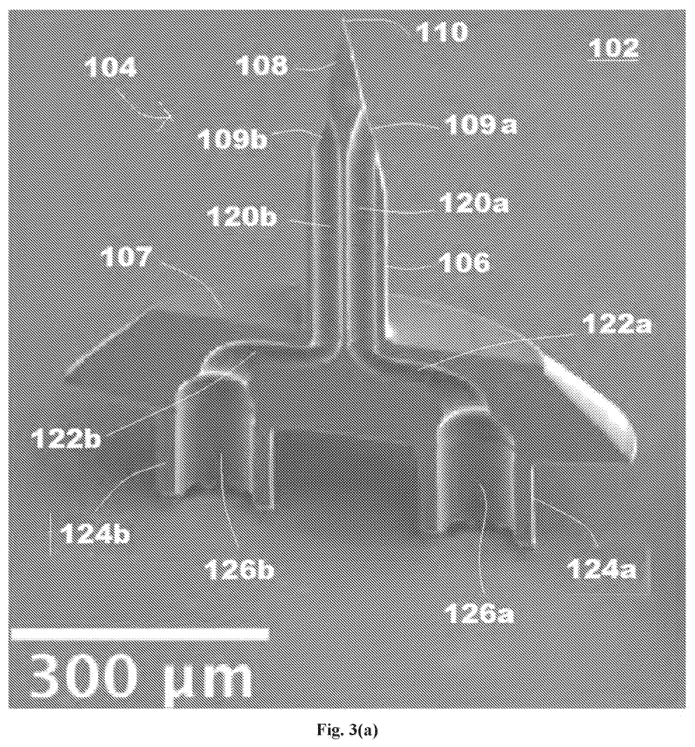

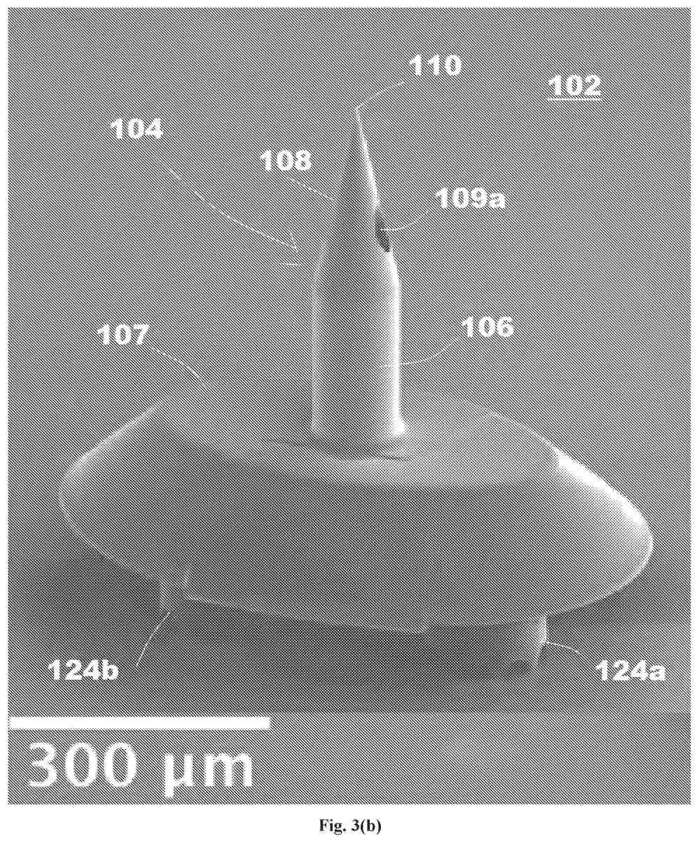

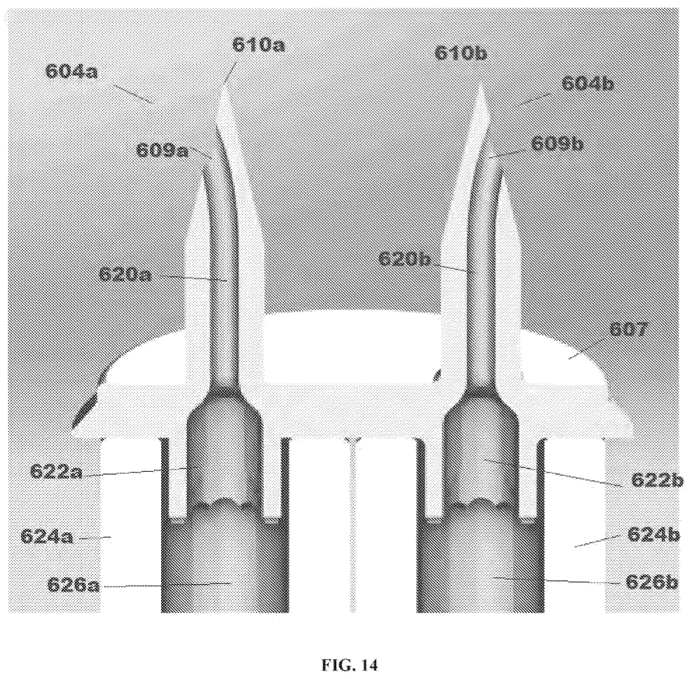

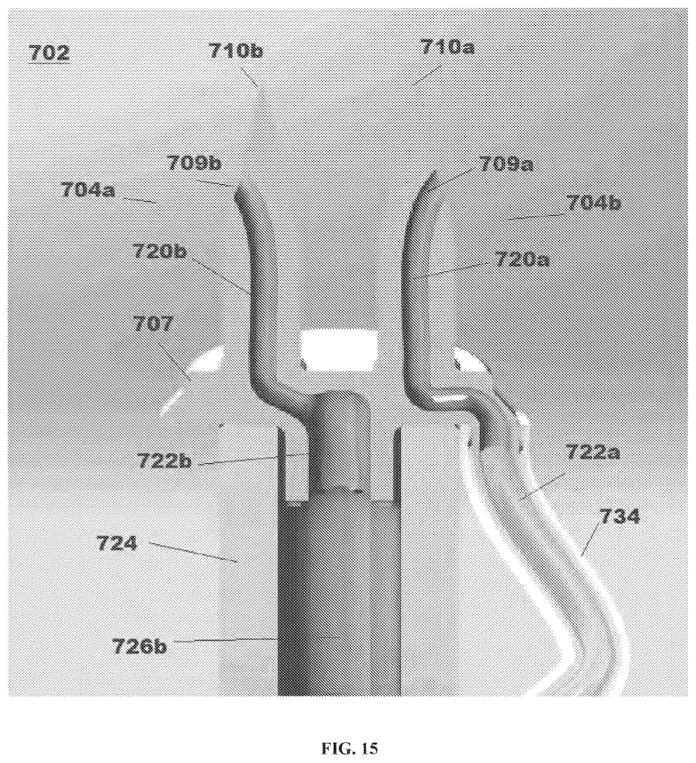

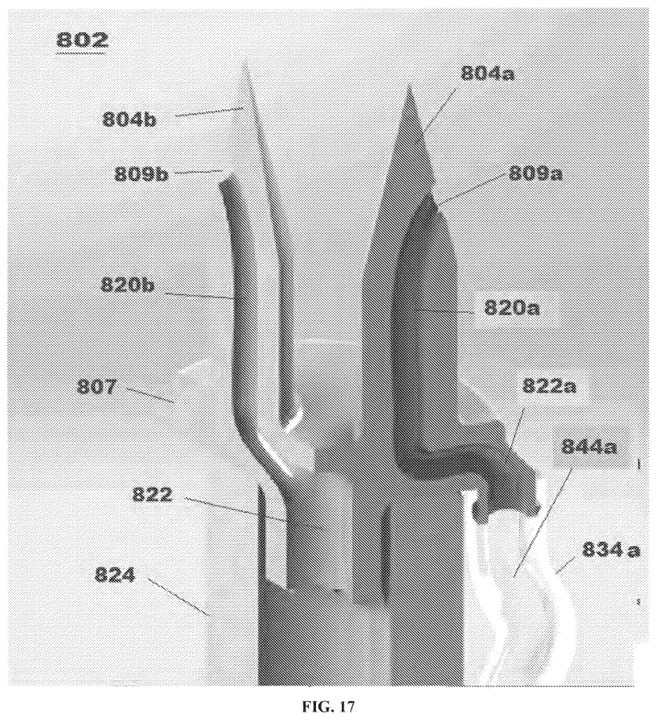

Microneedle. As illustrated in FIGS. 3(a)-(b), the microneedle system 100 includes a microneedle assembly 102 including microneedle 104. As illustrated in FIGS. 3(a)-(b), single microneedle 104 used. It is understood that an array of multiple microneedles, such as two microneedle (FIGS. 14-15), three microneedles (FIGS. 17-17), or more microneedles can be used in the microneedle assembly described herein. The microneedle 104 is fabricated in some embodiments using two-photon polymerization (2PP) lithography with an acrylic-based resin, such as negative-tone resins, IP photoresins, e.g., IP-S Photoresist. In some embodiments, biocompatible polymers, stainless steel, or titanium can be used to manufacture the microneedle. The microneedle 104 can be a hollow microneedle mounted on an enlarged base portion 107, a having a substantially cylindrical shaft 106, a substantially conical tapered portion 108 and a sharpened tip portion 110. The microneedle 104 can be provided with one or more interior lumens, including a first lumen 120a having an opening 109a proximal the tip 110 and a second lumen 120b having an opening 109b proximal the tip 110, each of lumens 120a/120b can be used for injection/introduction or aspiration of fluid. In some embodiments, the lumens 120a/b are in in fluid communication with an interior portion of the tubing connected to the syringe pump 105.

In some embodiments, the microneedle 104 is manufactured from ultra-high precision 3D molds made via 2PP lithography. Two-photon lithography can be used to manufacture molds for making thermoplastic microneedle arrays for drug delivery and fluid sampling across the anatomic membranes the ear, eye and the CNS such as the RWM. Since the precision of this manufacturing process is very high, very smooth ultra-sharp needles can be made that are specifically engineered to reduce insertion force, minimizing the damage to the tissue in question and any surrounding tissue. For example, hollow microneedles with a diameter of about 100 μm can be used to perforate the RWM without hearing loss; these perforations have been shown to heal completely within 48-72 hours. Perforations made by the microneedles described herein can be lens-shaped or slit-like in nature and in some cases are generated through separation rather than scission of membrane fibers.

In an exemplary embodiment, the shaft 106 has a diameter of 100 μm. In some embodiments, the shaft has a diameter in the range of 50 μm to 200 μm. Exemplary diameter dimensions of the shaft 106 of microneedle 104 include 50 μm, 60 μm, 75 μm, 90 μm, 100 μm, 110 μm, 125 μm, 1400 μm, 150 μm, 175 μm 200 μm and any dimensions inclusive. In an exemplary embodiment, the needle length is 475 μm. In some embodiments, the needle length is 250 μm, 275 μm, 300 μm, 350 μm, 400 μm, 450 μm, 475 μm, 500 μm, 550 μm, 600 μm, 650 μm, 700 μm, 750 μm and any dimensions inclusive. In an exemplary embodiment, the diameter of the interior lumens 120a/102b is 30 μm. In some embodiments, diameter of the interior lumen 109 is 15 μm, 20 μm, 30 μm, 40 μm, 50 μm, 60 μm, 70 μm, 80 μm and any dimensions inclusive. In an exemplary embodiment, the sharpness of the needle is defined by a tip radius of 500 nm to 3 μm. In some embodiments, the tip radius is 500 nm, 600 nm, 700 nm, 800 nm, 900 nm, 1 μm, 2 μm, 3 μm and any dimensions inclusive Further details regarding the microneedle are disclosed in applications WO/2014/093875; U.S. Pat. No. 10,821,276; WO/2015/20092; U.S. Pat. No. 11,413,191; U.S. application Ser. No. 17/887,966; WO/2017/160948; US 2019/0200927; WO/2019/136133; US 2020/0345994; WO/2019/204760; US 2021/0045925; US 2022/0175413; WO/2021/050404; US 2022/0176096; WO/2020/214802; US 2022/0032023, and PCT/US23/61545 all of which are incorporated by reference in their entirety herein.

Microneedle Assembly. With further reference to FIGS. 3(a)-(b), Microneedle 104 is mounted to needle base 107, which in turn is mounted to a pair of syringe needles 124a and 124b having a blunt end and affixed to base 107. In some embodiments, base 107 is fabricated from the same materials as the needle 104 and integral therewith, e.g., via 2PP photolithography using photoresist. Lumen 120a of microneedle 104 is in fluid communication with the interior lumen 126a of syringe needle 124a via a passage 122a in base 107. Similarly, lumen 120b is in fluid communication with the interior lumen 126b of syringe needle 124b via a passage 122a in base 107. Syringe needles 124a and 124b are secured to the tubing 151 for fluid communication with syringe pump 105 via tubing 151 and 153. In some embodiments, openings 109a and 109b are aligned with respect to the longitudinal dimension of the microneedle 104.

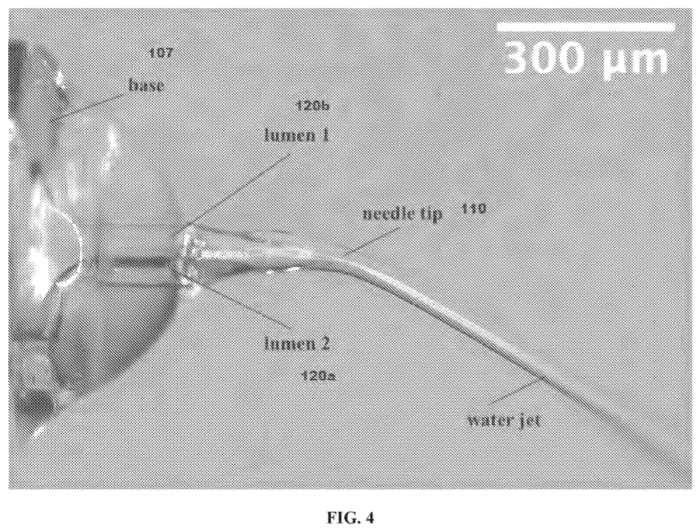

Methods. The microneedle system 100 disclosed herein was used for simultaneous aspiration and injection at a constant volume into the inner ear of a subject to facilitate delivery of a large volume of therapeutic agent. When used with deionized water, volumetric flow rates of up to 0.20 microliters per second for injection and 0.12 microliters per second for aspiration could be achieved without damaging the microneedle 104, corresponding to Reynolds number of 4.31 and 7.19 consistent with laminar flow. FIG. 4 is an optical microscope image of the dual lumen microneedle assembly 102 discussed herein and illustrated in FIGS. 3(a)-(b). One of the lumens 120a/b was pressurized by syringe pump 105. A thin stream of deionized water can be seen to jet out of the lumen.

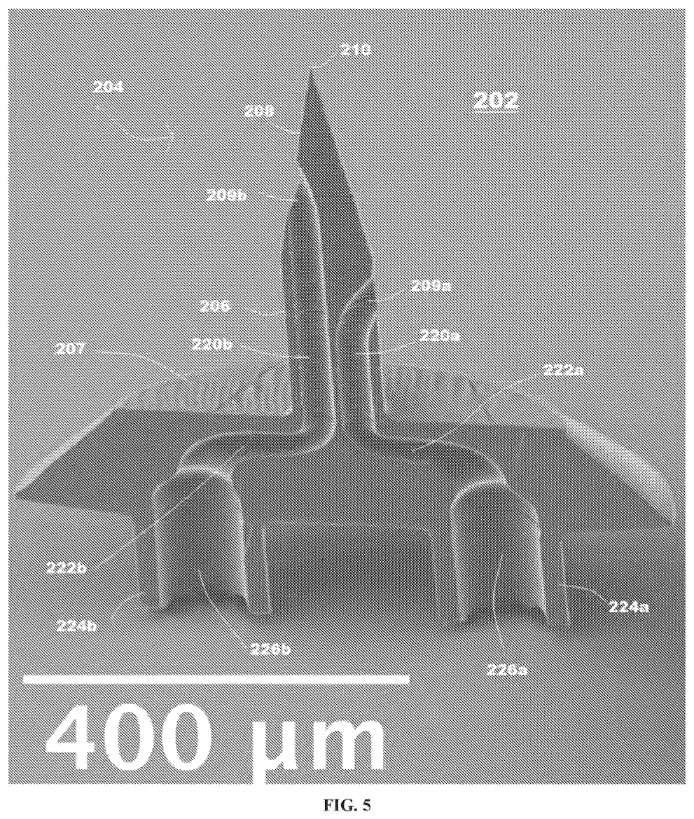

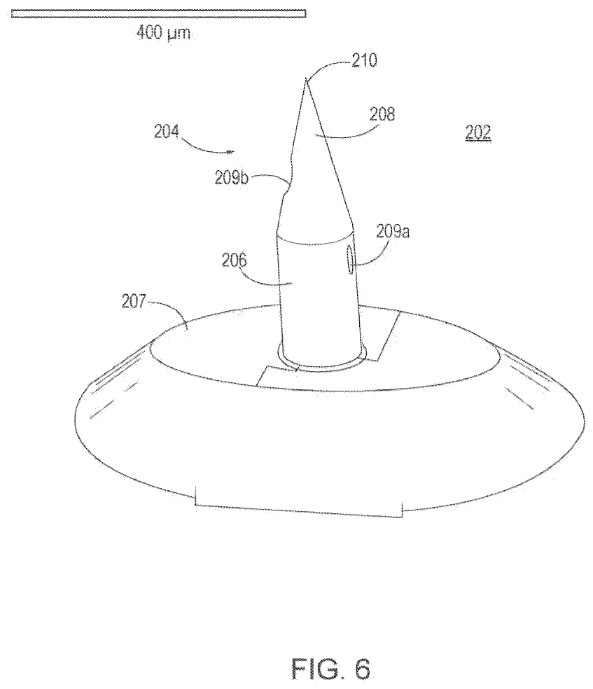

FIGS. 5-17 illustrate further embodiments of the microneedle assembly described herein. FIGS. 5-6 illustrate a second embodiment of the microneedle assembly 202, which is substantially identical to microneedle assembly 102, with the substantial differences noted herein. For example, lumen openings 209a and 209b are longitudinally offset. Microneedle 204 includes a first lumen 220a having a lumen opening 209a at the shaft portion 206 of the needle 24 and the second lumen 220b having a lumen opening 209b below the tip 210 in the conical portion 208 of the needle 204.

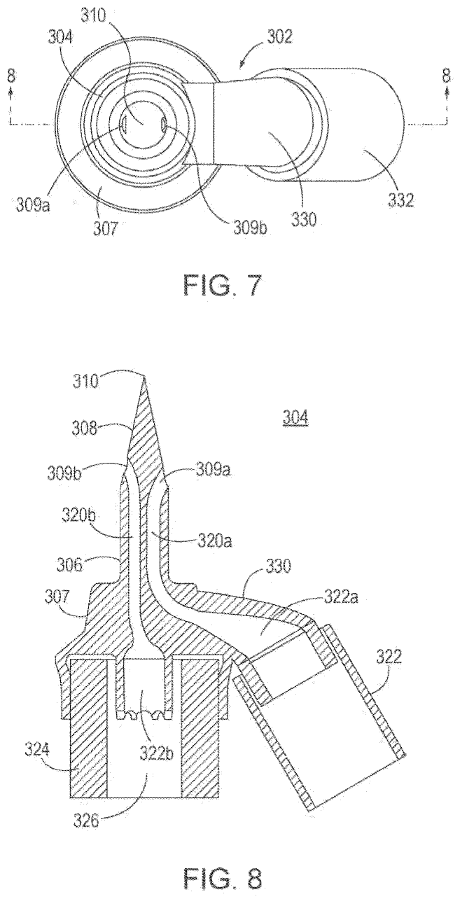

FIGS. 7-8 illustrate a third embodiment of the microneedle assembly 302, which is substantially identical to microneedle assembly 102, with the substantial differences noted herein. For example, the base 307 includes a portion 330 including an internal passage 322a which diverts the fluid from first lumen 320a to an external tube 332, e.g., polyimide tubing. Base 307 is mounted on a single blunt needle 324. The second lumen 320b is fluidly connected to the interior lumen 326 in syringe 324 by a passage 322b in base 307.

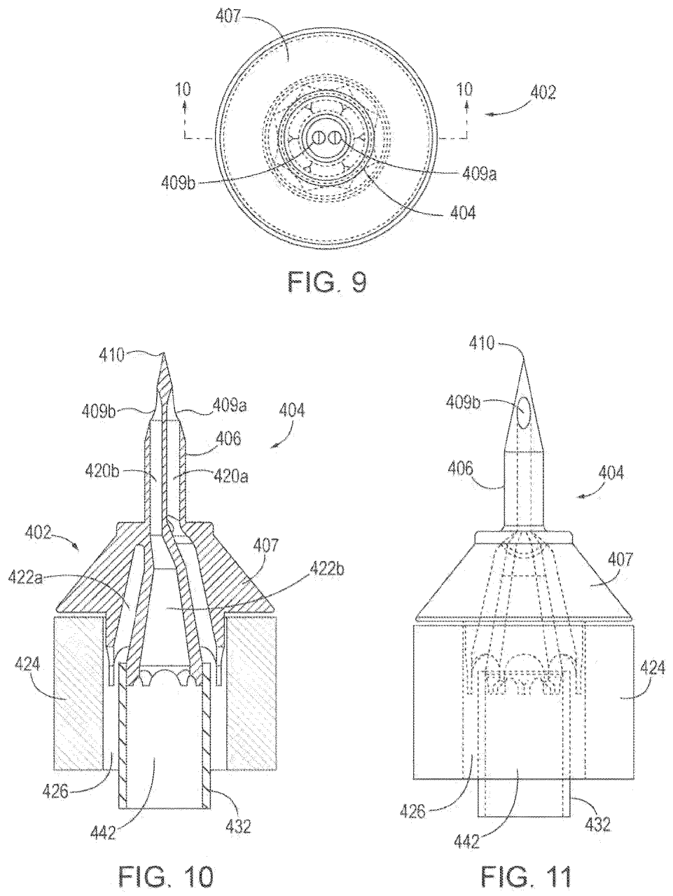

FIGS. 9-11 illustrate a fourth embodiment of the microneedle assembly 402, which is substantially identical to microneedle assembly 102, with the substantial differences noted herein. For example, base 407 is mounted on a single blunt needle 424. Base 407 includes two concentric passages 422a and 422b. Central passage 422b connects lumen 420b to an interior lumen 442 of tube 432, e.g., polyimide tubing. Outer concentric passage 422a connects lumen 420a to the lumen 426 of syringe 424, and as such fluid is communicated around tubing 432. In some embodiments, openings 409a and 409b are aligned with respect to the longitudinal dimension of the microneedle 404.

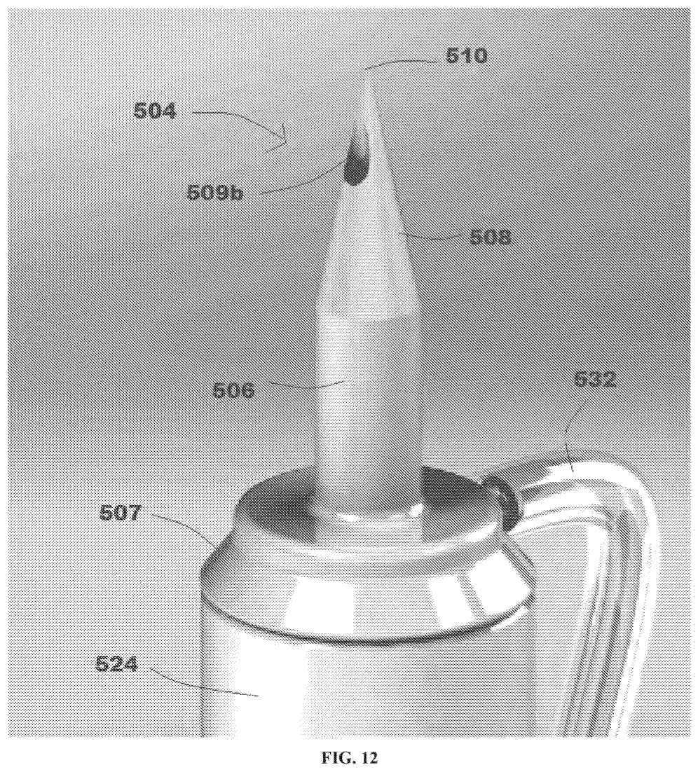

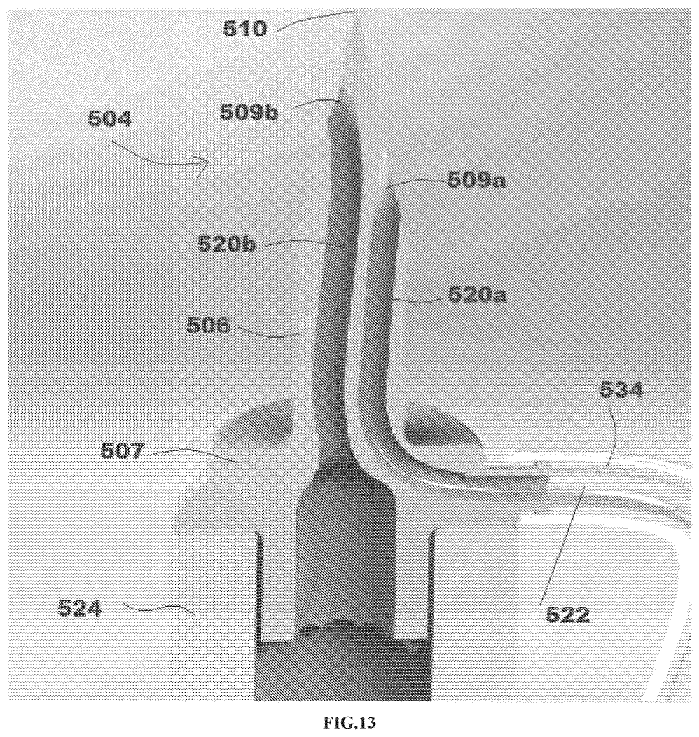

FIGS. 12-13 illustrate a fifth embodiment of the microneedle assembly 502, which is substantially identical to microneedle assembly 302, with the substantial differences noted herein. For example, microneedle 504 includes a first lumen 520a having a lumen opening 509a on first position of the tapered portion of the needle 504 and the second lumen 520b having a lumen opening 509b below the tip 210 in the tapered portion of the needle 504. Lumen opening 509a and 509b are located at offset positions along the length of the needle 504.

FIG. 14 illustrates a sixth embodiment of the microneedle assembly 602, which is substantially identical to microneedle assembly 102, with the substantial differences noted herein. For example, microneedle assembly 602 includes two microneedles 604a and 604b. Each of microneedles 604a and 604b includes a single lumen 620a and 602b and a single lumen opening 609a and 609b, respectively. The base 607 is mounted on two blunt syringe needles 624a and 624b. Lumen 620a is connected to the lumen 626a of needle 624a via a passage 622a in the base 607. Similarly, lumen 620b is connected to the lumen 626b of needle 624b via a passage 622b in the base 607.

FIG. 15 illustrates a seventh embodiment of the microneedle assembly 702, which is substantially identical to microneedle assembly 602, with the substantial differences noted herein. For example, microneedle assembly 702 includes two microneedles 704a and 704b. Each of microneedles 704a and 704b includes a single lumen 720a and 720b and a single lumen opening 709a and 709b, respectively. The base 707 is mounted on one blunt syringe needle 724. Lumen 720b is connected to the lumen 726b of needle 724 via a passage 722b in the base 707. The base 707 includes a passage which diverts the fluid from first lumen 720a to an external tube 734, e.g., polyimide tubing.

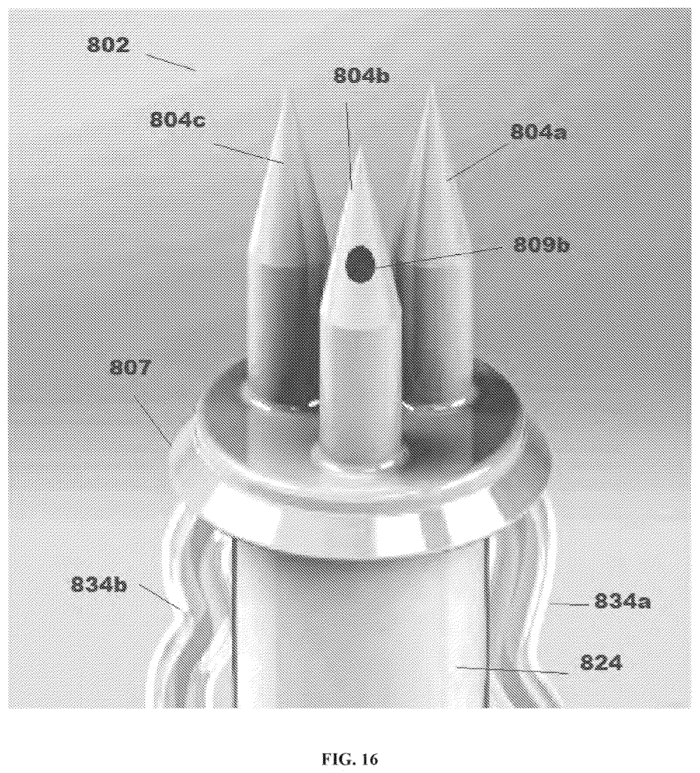

FIGS. 16-17 illustrates an eighth embodiment of the microneedle assembly 802, which is substantially identical to microneedle assembly 702, with the substantial differences noted herein. For example, microneedle assembly 802 includes three microneedles 804a, 804b and 804c. Each of microneedles 804a, 804b and 804c includes a single lumen 820a, 820b and 820c (not shown) and a single lumen opening 809a, 809b and 809c (not shown), respectively. The base 807 is mounted on one blunt syringe needle 824. Lumen 820b is connected to the lumen of needle 824 via a passage 822 in the base 807. The base 807 includes two passages 822a and 822c (not shown) which divert the fluid from lumen 820a and 802c (not shown) to an external tube 834a and 834, e.g., polyimide tubing.

While the work described herein focuses on accessing the cochlea, the technology can be translated to other anatomic barriers and enclosed spaces in the eye and central nervous system. Biodegradable ultra-sharp microneedles could be used to deliver therapeutic materials across the meninges into the brain and spinal cord, across the sclera into the eye and across the nerve sheath into peripheral nerves.

Various modifications, changes, and variations may be made in the arrangement, operation, and details of the methods and apparatus described above without departing from the scope of the claims.

What is claimed:

1. A microneedle system, comprising:
a needle assembly comprising:
a microneedle having a longitudinal body with a diameter of about 50 microns to about 200 microns, the microneedle defining a sharpened tip and a proximal end thereof, the microneedle having first and second lumens extending therethrough, each of the first and second lumens having a distal openings located proximal to the sharpened tip and a proximal opening at a proximal end of the microneedle;
a base having a distal end and a proximal end, the distal end of the base coupled to the proximal end of the microneedle and defining first and second passages in fluid communication with the first and second lumens; and
a pair of tubes coupled to the base, each tube in fluid communication with a respective first and second passage of the base;
a syringe pump; and
circulation tubing connecting the syringe pump with the pair of tubes in the needle assembly.

2. The microneedle system of claim 1, wherein the microneedle has a maximum outer diameter less than about 100 microns along a portion of the longitudinal body.

3. The microneedle system of claim 1, wherein the microneedle has a maximum outer diameter less than about 50 microns along a portion of the longitudinal body.

4. The microneedle system of claim 1, wherein the microneedle is fabricated from a biocompatible polymers, stainless steel, or titanium.

5. The microneedle system of claim 1, wherein the base is integral with the microneedle.

6. The microneedle system of claim 1, wherein the pair of tube comprising a blunt metallic syringe needle and a flexible polymer tubing.

7. The microneedle system of claim 6, wherein the flexible polymer tubing is fabricated from polyimide material.

8. The microneedle system of claim 6, wherein the blunt metallic syringe needle is a 30 gauge syringe needle.

9. The microneedle system of claim 1, wherein the pair of tube comprising two blunt metallic syringe needles.

10. The microneedle system of claim 1, wherein the first and second lumens have a diameter of about 15 microns to about 80 microns.

11. The microneedle system of claim 1, wherein the first and second lumens have a diameter of about 30 microns.

12. The microneedle system of claim 1, wherein fluid is aspirated into one of the first and second lumens at a flow rate of a maximum of 0.12 microliters per second.

13. The microneedle system of claim 1, wherein fluid is injected from one of the first and second lumens at a flow rate of a maximum of 0.20 microliters per second.

14. The microneedle system of claim 1, wherein the microneedle has a length of about 250 microns to about 750 microns.

15. The microneedle system of claim 1, wherein the microneedle has a length of about 475 microns.

16. The microneedle system of claim 1, wherein the first and second distal lumens opening are aligned along the length of the microneedle.

17. The microneedle system of claim 1, wherein the first and second distal lumen openings are offset along the length of the needle.

18. The microneedle system of claim 1, wherein the base defines first and second concentric passages for providing fluid communication between the first and second lumens and the pair of tubes.

19. A microneedle system comprising:
a needle assembly comprising:
a first microneedle having a longitudinal body with a diameter of about 50 microns to about 200 microns, the microneedle defining a sharpened tip and a proximal end thereof, the first microneedle having first lumen extending therethrough, the first lumen having a distal opening located proximal to the sharpened tip and a proximal opening at a proximal end of the first microneedle;
a second microneedle having a longitudinal body with a diameter of about 50 microns to about 200 microns, the second microneedle defining a sharpened tip and a proximal end thereof, the microneedle having second lumen extending therethrough, the second lumen having a distal opening located proximal to the sharpened tip and a proximal opening at a proximal end of the second microneedle;
a base having a distal end and a proximal end, the distal end of the base coupled to the proximal end of the first microneedle and the proximal end of the second microneedle, the base defining first and second passages in fluid communication with the first and second lumens; and
a pair of tubes coupled to the base, each tube in fluid communication with a respective first and second passage of the base;
a syringe pump; and
circulation tubing connecting the syringe pump with the pair of tubes in the needle assembly.

20. The microneedle system of claim 19, wherein the first and second microneedles have a maximum outer diameter less than about 100 microns along a portion of the longitudinal body.

21. The microneedle system of claim 19, wherein the first and second microneedles have a maximum outer diameter less than about 50 microns along a portion of the longitudinal body.

22. The microneedle system of claim 19, wherein the first and second microneedles are fabricated from a biocompatible polymers, stainless steel, or titanium.

23. The microneedle system of claim 19, wherein the base is integral with first and second microneedles.

24. The microneedle system of claim 19, wherein the pair of tubes comprising a blunt metallic syringe needle and a flexible polymer tubing.

25. The microneedle system of claim 24, wherein the flexible polymer tubing is fabricated from polyimide material.

26. The microneedle system of claim 24, wherein the blunt metallic syringe needle is a 30 gauge syringe needle.

27. The microneedle system of claim 19, wherein the pair of tubes comprising two blunt metallic syringe needles.

28. The microneedle system of claim 19, wherein the first and second lumens each have a diameter of about 15 microns to about 80 microns.

29. The microneedle system of claim 19, wherein the first and second lumens each have a diameter of about 30 microns.

30. The microneedle system of claim 19, wherein fluid is aspirated into one of the first and second lumens at a flow rate of a maximum of about 0.15 microliters per second.

31. The microneedle system of claim 19, wherein fluid is injected from one of the first and second lumens at a flow rate of a maximum of about 0.2 microliters per second.

32. The microneedle system of claim 19, wherein the first and second microneedles have a length of about 250 microns to about 750 microns.

33. The microneedle system of claim 19, wherein the first and second microneedles have a length of about 475 microns.

34. The microneedle system of claim 19, wherein the microneedle assembly further comprises a third microneedle having a longitudinal body with a diameter of about 50 microns to about 200 microns, the third microneedle defining a sharpened tip and a proximal end thereof, the third microneedle having third lumen extending therethrough, the third lumen having a distal opening located proximal to the sharpened tip and a proximal opening at a proximal end of the third microneedle; wherein the base coupled to the proximal end of the third microneedle and defining a third passages in fluid communication with the third lumens.
